(19) United States
(12) Patent Application Publication
(10) Pub. No.: US 2010/0310456 A1
(43) Pub. Date: Dec. 9, 2010

(54) IMAGING OF MYELIN BASIC PROTEIN

(75) Inventors: Tiberiu Mircea Siclovan, Rexford, NY (US); Bruce Fletcher Johnson, Scotia, NY (US); Rong Zhang, Niskayuna, NY (US); Cristina Abucay Tan Hehir, Niskayuna, NY (US)

Correspondence Address: GENERAL ELECTRIC COMPANY;GLOBAL RESEARCH, ONE RESEARCH CIRCLE, BLDG. K1-3A59, NISKAYUNA, NY, 12309, US

(73) Assignee: GENERAL ELECTRIC COMPANY, Schenectady, NY (US)

(21) Appl. No.: 12/694,820

(22) Filed: Jan. 27, 2010

Related U.S. Application Data
(63) Continuation of application No. 12/478,300, filed on Jun. 4, 2009.

(51) Int. Cl. A61K 51/00; A61K 49/00

(52) U.S. Cl. 424/1.81; 424/9.1; 424/9.3

(57) ABSTRACT

The present invention relates to methods for myelin basic protein detection comprises identifying a subject at risk of or diagnosed with a myelin-associated neuropathy, parenterally administering to the subject the agent, and determining myelination in the subject by detecting binding to myelin basic protein. Methods for the detection of myelin and a quantitative measurement of its local concentration in a sample using an agent with specific binding to myelin basic protein are also provided as is a kit containing the agent or its derivatives for use in detecting myelin basic protein

### CROSS-REFERENCE TO RELATED APPLICATIONS

This application is a continuation-in-part to U.S. patent application Ser. No. 12/478,300 filed Jun. 4, 2009; the disclosure of which is incorporated herein by reference in its entirety.

### BACKGROUND

Information flow within the nervous system requires the perpetuation of ionic gradients along neurons. In many neurons, effective and efficient perpetuation of such gradients along axons requires electrical insulation. Myelin, a lipid-rich, dielectric substance that ensheathes axons, serves this insulating function. The nervous system contains high levels of myelin, which is especially enriched where many myelinated axons are bundled together, such as in tracts of the spinal cord and spinal nerve roots, nerves in the peripheral nervous system, and fiber tracts in the brain, collectively called “white matter”, as opposed to “grey matter”. Because non-nervous system tissue lacks myelin, the presence of myelin can distinguish nerve tissue from other tissue types; the spinal cord and spinal nerve roots from non-nervous elements of the vertebral column, and white matter from grey matter in the brain.

The ability to qualitatively or quantitatively visualize myelin, either in vivo or in vitro, confers upon researchers and clinicians important diagnostic and treatment tools. For example, the ability to visually identify peripheral nerves during surgery assists surgeons in avoiding cutting or damaging nerves. Previous efforts in image-guided surgery of nerves utilized modalities that would not require contrast agents or fluorescent labeling of axons by retrograde transport. A challenge in the first approach is that the signal is typically ambiguous

Retrograde labeling of nerves in animal models is widely reported in the literature. Although this strategy may work, there are many inherent problems. Labeling would depend on exactly where the contrast agent is injected. If the nerves fail to take up the contrast agent, the nerve would not be visualized. In some cases, nerve stimulation is required to facilitate retrograde transport. The long times required for retrograde transport may not be clinically feasible.

Myelinated nerves and fiber tracts serve as useful landmarks in anatomical studies carried out by preclinical and basic neuroscience researchers. Furthermore, the formation of myelin sheaths is an important step in the generation and functional stability of new neurons; so the availability of myelin markers may aid researchers study such processes. Myelin-labeling methodologies are also useful in the development of numerous therapies, neural stem cell research, and putative animal models of myelin-associated neuropathies. In vivo myelin imaging of the spinal cord assists clinicians in the diagnosis and treatment of spinal cord pathology, such as nerve compression or herniated discs as well as myelin-associated neuropathies, such as multiple sclerosis which results in damage to myelin within the central or peripheral nervous system. The ability to measure amounts of myelination in vivo in patients with such conditions would aid clinicians and researchers in diagnosing and progno sing myelin-associated neuropathies.

The spinal nerve roots can be damaged as they traverse the spinal canal, but are especially vulnerable in the intervertebral foramen, where the spinal nerve roots join to form the spinal nerves. Syndromes such as cervical radiculopathy, sciatica, intervertebral disc herniation, and root compression are caused by compression primarily from tumors or other lesions, which usually present with back or neck pain. Back or neck pain may be caused by a variety of musculoskeletal mechanisms and the physician needs to be able to examine the nervous system to determine if there is compression of nerve roots or the spinal cord. The ability to image and identify the source of chronic neck or back pain could enable surgeons to effectively treat these syndromes.

Myelin-labeling methodologies do exist, including the use of commercially available FluoroMyelin dyes for identification of high myelin content tissues. However, except for a few dyes such as bis-styrene-arylene dyes such as 1,4-bis(p-aminostyryl)-2-methoxy benzene (BMB), and (E,E)-1,4-bis(4′-aminostyryl)-2-dimethoxy-benzene (BDB), most of the publicly-disclosed dyes are unable to cross the blood nerve or blood brain barrier.

Myelin is a protein and lipid-rich matrix formed by oligodendrocytes in the central nervous system (CNS) and Schwann cells in the peripheral nervous system (PNS). Because two different cell types in CNS and PNS produce myelin, namely oligodendrocytes and Schwann cells respectively, there are similarities and differences in protein and lipid composition depending on the source of myelin. In both instances, myelin is composed of about 80% lipid fraction and about 20% protein fraction. Numerous studies have examined the molecular components of both fractions.

The lipid fraction in myelin contains cholesterol, cholesterol ester, cerebroside, sulfatide, sphingomyelin, phosphotidylethanolomine, phosphotidylcholine, phosphotidylserine, phosphotidylinositol, triacylglycerol, and diacylglycerol. The protein fraction is composed of several proteins, which include myelin basic protein (MBP), peripheral myelin protein 22 (PMP22), connexin 32 and myelin-associated glycoprotein (MAG), which are, produced by both PNS and CNS cells; the protein myelin protein zero (MPZ), produced by the PNS only; and proteolipid protein, produced by CNS cells only.

MBP is a major protein component of myelin at 5%-15%, which translates into about 5 mM concentration of MBP. Techniques such as circular dichroism, NMR and EPR spectroscopy, atomic force microscopy and others, suggest that MBP may have a compact C-shaped form with a core element of beta-sheet structure, but only when associated with lipids. The interaction of myelin basic protein to lipids can cause conformational variability and may be critical for function.

An agent that selectively binds to MBP may result in improvements in myelin staining and thereby aid in nerve visualization. Nerve visualization my be further improved through, optimal elimination of unbound and nonspecifically bound dye, and improved optical properties to allow enhanced contrast between myelin and surrounding tissue. Optical properties in the near infrared range (NIR), between 700-900 nm, are ideal for visualization of myelin in vivo. In the NIR range the absorption of water, hemoglobin, and lipid are minimal, and scatter is reduced such that photon penetration is improved. Also, autofluorescence is low and the NIR light penetrates deep into tissue and is less affected by scatter.

### BRIEF DESCRIPTION

Provided herein are methods for the detection of myelin-associated neuropathy comprising identifying a subject at risk of or diagnosed with a myelin-associated neuropathy, administering to a subject an agent that binds specifically to myelin basic protein, and determining myelination in the subject by detecting the agent present in the subject.

In one embodiment the agent comprises a compound of Formula I, a ¹³C enriched compound of Formula I, an ¹⁹F-labeled derivative of Formula I, or a radioisotope derivative of Formula I

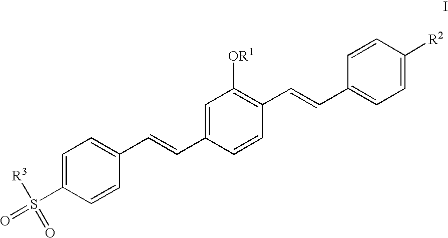

wherein R¹ is an alkyl group, R² is an electron donating group, and R³ is an alkyl, substituted alkyl, amine or substituted amine.

In one embodiment a method of imaging myelin basic protein in a surgical field is provided comprising the steps of: contacting the surgical site with an agent, wherein the agent comprises a compound of Formula I, a 13C enriched compound of Formula I, an 19F-labeled derivative of Formula I, or a radioisotope derivative of Formula I, and detecting the agent.

In another embodiment a kit for detecting myelin-associated neuropathy in a subject is provided, the kit comprising an agent at binds specifically to myelin basic protein and a pharmaceutically acceptable carrier.

### BRIEF DESCRIPTION OF THE FIGURES

These and other features, aspects, and advantages of the present invention will become better understood when the following detailed description is read with reference to the accompanying figures wherein:

FIG. 1 shows results from ex vivo staining of rat femoral nerve (top panel), sciatic (middle) and trigeminal nerve sections (bottom) by Formula Ia compounds.

FIG. 2 shows results from fluorescence in vivo imaging of the nerves in the brachial plexus of a mouse treated with Formula I (R1=CH₃, R²═NH₂ and R³═—CH₃).

FIG. 3 shows a Spectramax M5 assay on Formula Ia (R1=CH₃, R²═NH₂ and R³═—CH₃) and Formula Ia (R1=CH₃, R²═NH₂ and R³═—CF₃) in the presence and absence of purified native-like MBP or denatured MBP Formula Ia (R1=CH₃, R²═NH₂ and R³═—CH₃) was excited at 400 nm, and with fluorescence emission intensity read at 610 nm. Formula I (R1=CH₃, R²═NH₂ and R³═—CF₃) was excited at 430 nm, and with fluorescence emission intensity read at 630 nm.

### DETAILED DESCRIPTION

The following detailed description is exemplary and not intended to limit the invention of the application and uses of the invention. Furthermore, there is no intention to be limited by any theory presented in the preceding background of the invention or descriptions of the drawings.

### Definitions

To more clearly and concisely describe and point out the subject matter of the claimed invention, the following definitions are provided for specific terms, which are used in the following description and the appended claims.

“Myelin-associated neuropathy” generally refers to any condition in which the insulating material ensheathing portions of neuronal cells becomes damaged or dysfunctional as a component of a syndrome, disease, or other pathological condition, such as, but not limited to, multiple sclerosis, Guillain-Barré syndrome, leukodystrophies, metachromatic leukodystrophy, Refsum' s disease, adrenoleukodystrophy, Krabbe's disease, phenylketonuria, Canavan disease, Pelizaeus-Merzbacher disease, Alexander's disease, diabetic neuropathy, chemotherapy induced neuropathy, or any combination thereof.

“Agent” refers to a solution or carrier for introducing a compound into a subject in a manner to allow the compound to be administered at a desired concentration and efficacy. The agent may include, but is not limited to, solvents, stabilization aids, buffers, and fillers.

An agent exhibits “specific binding” for myelin if it associates more frequently with, more rapidly with, for a longer duration with, or with greater affinity to, myelin than with tissues not containing myelin. “Non-specific binding” refers to binding of the agent to non-myelin containing tissue. For relative binding values, such as specific binding or non-specific binding, each sample should be measured under similar physical conditions (i.e., temperature, pH, formulation, and mode of administration). Generally, specific binding is characterized by a relatively high affinity of an agent to a target and a relatively low to moderate capacity. Typically, binding is considered specific when the affinity constant Kₐ is at least 10⁶ M⁻¹. A higher affinity constant indicates greater affinity, and thus typically greater specificity. For example, antibodies typically bind antigens with an affinity constant in the range of 10⁶ M⁻¹ to 10⁹ M⁻¹ or higher. “Non-specific” binding usually has a low affinity with a moderate to high capacity. Non-specific binding usually occurs when the affinity constant is below 10⁶ M⁻¹. Controlling the time and method used to contact the agent with the tissues reduces non-specific binding.

“Washing” generally refers to any method, such as but not limited to, immersion in, or flushing by repeated application of, a non-labeling solution or other substance, such as but not limited to water, saline, buffered saline, or ethanol, so as to provide a medium for dissociation, dispersal, and removal of unbound or non-specifically bound labeling compound from non-myelinated components of the tissue or sample of tissue without eliminating specific binding to myelin.

“Baseline fluorescence” refers to the frequency and magnitude of electromagnetic radiation emitted by a tissue or sample of tissue upon being exposed to an external source of electromagnetic radiation in the absence of administration or binding of any fluorescing compound, as distinguished from the radiation emitted following the administration and binding of such fluorescing compound and exposure to an external source of electromagnetic radiation.

“Control sample representative of the tissue section” refers to a tissue sample of a similar size, morphology, or structure as the tissue sample to be analyzed, and with a level of myelin whereby the sample's level of myelin serves as a reference to which other samples' myelin levels may be compared.

“Parenteral administration” refers to any means of introducing a substance or compound into a subject, that does not involve oral ingestion or direct introduction to the gastrointestinal tract, including but not limited to subcutaneous injection, intraperitoneal injection, intramuscular injection, intravenous injection, intrathecal injection, intracerebral injection, intracerebroventricular injection, intraspinal injection, intrathecal injection, intracerebral injection, intracerebroventricular injection, or intraspinal injection or any combination thereof.

“Pharmaceutical carrier” refers to a composition which allows the application of the agent material to the site of the application, surrounding tissues, or prepared tissue section to allow the agent to have an effective residence time for specific binding to the target or to provide a convenient manner of release. Solubilization strategies may include but are not limited to: pH adjustments, salt formation, formation of ionizable compounds, use of co-solvents, complexation, surfactants and micelles, emulsions and micro-emulsions. The pharmaceutical carrier may include, but is not limited to, a solubilizer, detergent, buffer solution, stabilizers, and preservatives. Examples of these include but are not limited to, HCl, citric acid, DMSO, propylene glycol, ethanol PEG 300, cyclodextrans, citrate, acetate, phosphate, carbonate or tris (hydroxymethyl)amino methane.

“Demyelination model” refers to any experimentally-induced damage to, or dysfunction of, the insulating material ensheathing portions of neuronal cells, that may be utilized in the experimental study of neuropathic demyelination, including, but not limited to, experimental allergic encephalomyelitis.

“Remyelination” refers to the spontaneous, therapeutic, or experimentally induced repair, regeneration, or otherwise enhanced constitution or functionality of the insulating material ensheathing neuronal axons.

“Alkyl” is intended to include linear, branched, or cyclic hydrocarbon structures and combinations thereof, including lower alkyl and higher alkyl. Alkyl groups are those of C20 or below. “Lower alkyl” refers to alkyl groups of from 1 to 6 carbon atoms, preferably from 1 to 4 carbon atoms, and includes methyl, ethyl, n-propyl, isopropyl, and n-, s- and t-butyl. Higher alkyl refers to alkyl groups having seven or more carbon atoms, preferably 7-20 carbon atoms, and includes n-, s- and t-heptyl, octyl, and dodecyl. Cycloalkyl is a subset of alkyl and includes cyclic hydrocarbon groups of from 3 to 8 carbon atoms. Examples of cycloalkyl groups include cyclopropyl, cyclobutyl, cyclopentyl, and norbornyl. Alkenyl and alkynyl refer to alkyl groups wherein two or more hydrogen atoms are replaced by a double or triple bond, respectively.

“Substituted” refers to residues, including, but not limited to, alkyl, alkylaryl, aryl, arylalkyl, and heteroaryl, wherein up to three H atoms of the residue are replaced with lower alkyl, substituted alkyl, aryl, substituted aryl, haloalkyl, alkoxy, carbonyl, carboxy, carboxalkoxy, carboxamido, acyloxy, amidino, nitro, halo, hydroxy, OCH(COOH)₂, cyano, primary amino, secondary amino, acylamino, alkylthio, sulfoxide, sulfone, phenyl, benzyl, phenoxy, benzyloxy, heteroaryl, or heteroaryloxy.

“Electron donating group” refers to chemical groups that add electron density to the conjugated π system making it more nucleophilic. Electron donating groups may be recognized by lone pairs of electrons on an atom adjacent to the π system. Examples of electron donating groups include, but are not limited to, —NR′R″, —NHR, —NH₂, —OH, —OR, —NHCOR, —OCOR, —R, —C₆H₅, and —CH═CR₂.

“Electron withdrawing group” refers to chemical groups that remove electron density from the conjugated π system rendering the structure less nucleophilic. Electron withdrawing groups may be recognized either by the atom adjacent to the π system having several bonds to more electronegative atoms or, having a formal positive charge. Examples of electron withdrawing groups include, but are not limited to, —COH, —COR, —COOR, —COOH, —COONH₂, —COONHR, —COONR₂, —COCl, —CF₃, —CN, C═C(CN)₂ —SO₃H, —NH₃+, —NR₃+, —NO_{2,} —SO₂R, —SO₂NH_{2,} —SO₂NHR, and —SO₂NR₂.

An agent exhibits “specific uptake” for myelinated tissues if it associates more frequently with, more rapidly with, for a longer duration with, or with greater affinity to, or if it is absorbed more, or accumulates more in myelinated tissues than with non-myelinated tissues. Generally, specific uptake is characterized by a relatively high affinity of an agent to a target.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Many of the compounds described herein may comprise one or more asymmetric centers and may thus give rise to enantiomers, diastereomers, and other stereoisomeric forms that may be defined, in terms of absolute stereochemistry, as (R)- or (S)-. The chemical structure of the agent includes for example, without limitation, all such possible isomers, as well as, their racemic and optically pure forms. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also included.

In certain embodiments, methods for the qualitative or quantitative detection of myelin basic protein in an in vitro or in vivo sample utilizing specific binding of an agent to myelin basic protein is provided. The specific binding to myelin basic protein may be by an agent comprising the compound of Formula I, a ¹³C enriched compound of Formula I, an ¹⁹F-labeled-derivative of Formula I, or a radioisotope derivative of Formula I,

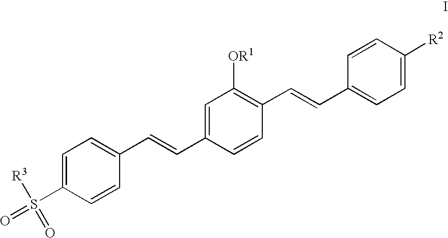

wherein R¹ is an alkyl group, R² is an electron donating group and R³ is an alkyl, substituted alkyl, amine, or substituted amine group

In certain embodiments R¹ may be a lower alkyl groups of from 1 to 6 carbon atoms, preferably from 1 to 4 carbon atoms, and includes methyl, ethyl, n-propyl, isopropyl, and n-, s- and t-butyl. The electron donating group may include a primary, secondary, or tertiary amine, or an alkoxy group.

In certain embodiments, R³ may be lower alkyl groups of from 1 to 6 carbon atoms, preferably from 1 to 4 carbon atoms, and includes methyl, ethyl, n-propyl, isopropyl, and n-, s- and t-butyl.

In other embodiments, R3 may be used to improve water solubility and reduce log P of the resulting sulfone. R3 may be a substituted alkyl group, such as, but not limited to an alkoxy or alcohol. In certain embodiments, the alkoxy group may contain ethylene glycol units or an ethylene glycol terminated alcohol. I For example R3 may be (CH₂CH₂O)nX or CH₂CH₂CH₂ (OCH₂CH₂)nOX where n is an integer between 1 and 6 and X is hydrogen, methyl or ethyl. The incorporation of a propyl group may also eliminate the potential for β elimination

In certain other embodiments, R³ may be a primary, secondary, or tertiary amine to form a sulfonamide. The amine groups include, but are not limited to NH₂, NHR⁴ and NR⁴R⁵ wherein R⁴ and R⁵ are alkyl or substituted alkyl groups. R⁴ and R⁵ may or may not be equivalent and may form a ring structure. For example R⁴ and R⁵ may be (CH₂CH₂O)ₙX, or CH(CH₂OX)₂, C(CH₂OX)₃ where n is an integer between 1 and 6 and X is hydrogen, methyl, or ethyl. In other examples R⁴ and R⁵ may from a ring structure such as a substituted piperidine, piperazine, or morpholine.

In each embodiment, R² and R³ SO₂ are conjugated through the π double bond orbitals of the benzene rings and olefinic substituents, thereby providing a clear path for electrons to flow from the electron donating group to the electron withdrawing group.

This conjugation and “push-pull” electron flow from R² to R³SO₂ may be responsible for a Stokes shift of a longer wavelength during fluorescence as compared to BMB and BDB. In applications, this may allow enhanced contrast between myelin and surrounding tissue when using an agent of Formula I.

In some embodiments, the agent, which specifically binds to myelin basic protein, may be a radioisotope, a ¹³C enriched compound, or an ¹⁹F-labeled derivative. In some embodiments, a radioisotope derivative of the compound of Formula I may be prepared and imaging accomplished through radioimaging. Alternatively, a ¹³C enriched compound of Formula I, or an ¹⁹F-labeled derivative of Formula I may be prepared.

The agent comprising the compound of Formula I, a ¹³C enriched compound of Formula I, an ¹⁹F-labeled-derivative of Formula I, or a radioisotope derivative of Formula I, may be detected by its emitted signal, such as a magnetic resonance signal or emitted radiation from a radioisotope derivative of Formula I, autofluorescence emission, or optical properties of the agent. The method of detection of agent comprising the compound of Formula I, a ¹³C enriched compound of Formula I, an ¹⁹F-labeled-derivative of Formula I, or a radioisotope derivative of Formula I, may include fluorescence microscopy, laser-confocal microscopy, cross-polarization microscopy, nuclear scintigraphy, positron emission tomography (“PET”), single photon emission computed tomography (“SPECT”), magnetic resonance imaging (“MRI”), magnetic resonance spectroscopy (“MRS”), computed tomography (“CT”), or a combination thereof, depending on the intended use and the imaging methodology available to the medical or research personnel.

For example, in certain embodiments, the R³ group of Formula I may be a fluoroalkyl such as —CF₃, —CH₂CF₃, or —OC(CF₃)₃ for the purpose of MRI imaging. In other examples R³ may be, —(CH₂CH₂O)ₙQ or CH₂CH₂CH₂O(CH₂CH₂O)ₘQ where n is an integer between 1 and 5, m is an integer between 0 and 4, and Q is CH₂CF₃, CH(CF₃)₂, or C(CF₃)₃.

Similarly where R³ may be a secondary, or tertiary amine to form a sulfonamide, the amine group may be substituted with a fluoroalkyl. In certain embodiments, R³ may be NHR⁴ or NR⁴ R⁵ where R⁴ and R⁵ may or may not be equivalent and equal —CH₂CF_{3,} or —(CH₂CH₂O)ₙQ, where n is an integer between 1 and 6 and Q is equal to CH₂CF₃, CH(CF₃)₂, or C(CF₃)₃. NR⁴R⁵ may also form a ring structure such as fluoroalkyl or fluoroalkoxyl substituted piperidine, piperazine, or morpholine.

For imaging methods using PET imaging, ¹⁸F radioisotopes may be incorporated into Formula I through its R¹, R² or R³ substituents. In certain embodiments, the ¹⁸F radioisotopes may be incorporated into the R³ substituent as described in the example above for the ¹⁹F-labeled-derivatives used in MRI imaging.

The imaging methods described may be applicable to analytical, diagnostic, or prognostic applications related to myelin basic protein detection. The applications may be particularly applicable in intraoperative nerve labeling, spinal imaging, brain tissue imaging, non-invasive in vivo measurement of myelination levels, and preclinical and basic neuroscience bench research aimed at the study of the function and process of myelination, and the dysfunction and repair of myelin.

In one embodiment, an agent which binds specifically to myelin basic protein may be administered parenterally to a surgical subject prior to surgery such that the agent binds to myelin basic protein and may be cleared from tissues that do not contain myelin basic protein. In another embodiment, the agent may be applied directly, via painting on, spraying on, or local injection to the surgical field during surgery, allowed to bind to myelin basic protein present, and the surgical site washed by lavage to clear unbound composition from the site. During surgery, a light source tuned to the spectral excitation characteristics of the agent may be applied to the surgical field. The agent may be observed through an optical filter tuned to its spectral emission characteristics. Due to their specific binding to the fluorescing agent, nerves and other myelin containing tissue are distinguishable from tissue not containing myelin basic protein. This enables the surgeon to avoid inadvertently cutting or damaging myelinated tissue by avoiding fluorescing tissue, or facilitates accurately administering treatment to the intended myelinated tissue. In certain embodiments the agent comprises the compound of Formula I.

An agent which specifically binds to myelin basic protein may be administered parenterally to a subject prior to surgery or prior to treatments targeting a nerve or other myelin containing tissue, such as pharmaceutical or surgical nerve block. In certain embodiments the myelinated tissue may be part of the spinal canal and intervertebral foramen. In other embodiments the myelinated tissue may be part of the brain. In certain embodiments the agent comprises the compound of Formula I, a ¹³C enriched compound of Formula I, an ¹⁹F-labeled-derivative of Formula I, or a radioisotope derivative of Formula I

In one embodiment an agent, such as one comprising the compound of Formula I, a ¹³C enriched compound of Formula I, or an ¹⁹F-labeled-derivative of Formula I, may be administered parenterally to a surgical subject, prior to surgery, to permit binding to myelin basic protein, and clearance from tissues that do not contain myelin basic protein without the elimination of specific myelin basic protein binding.

In another embodiment, an agent, which is a radioisotope and which specifically, binds to myelin basic protein may be administered parenterally to a subject prior to treatment to allow binding and clearance from tissues that do not contain myelin. Imaging techniques such as nuclear scintigraphy, PET, SPECT, CT, MRI, MRS, or any combination thereof, may then be used to aid in differentiation of the myelin and non-myelin containing tissues and may employ a gamma camera, a scanner or a probe. The agent may be a radioisotope derivative of the compound of Formula I

In another embodiment an agent, such as one comprising the compound of a radioisotope derivative of Formula I, may be administered parenterally to a patient suspected of, or determined to be, suffering from a spinal pathology, such as but not limited to, spinal compression, spinal nerve root compression, or a bulging disc. After binding to spinal myelin basic protein, and clearance from tissue that does not contain myelin basic protein without eliminating the specific myelin basic protein binding, the spine may be imaged for in vivo using radioisotope imaging such as PET, SPECT, or any combination thereof.

By inspection of the diagnostic images, the clinician may determine if, and where, the spinal cord, or associated nerve roots, are impinged, such as by the vertebral column or foreign matter. Additional scans, such as CT or MRI, may also be conducted in conjunction with PET or SPECT scans, to provide additional information, such as the structure and relative positioning of elements of the vertebral column. In one embodiment, this method may be applied to a surgical procedure to image the spinal region intraoperatively.

In another embodiment, myelination level is accessed in vivo by imaging a radioisotope derivative of an agent, which binds specifically to myelin basic protein. The agent is administered parenterally to a subject diagnosed with, or suspected of having, a myelin-associated neuropathy. After binding to myelin basic protein, and clearance from tissue that does not contain myelin basic protein without eliminating specific myelin basic protein binding, components of the central or peripheral nervous system may be imaged by a method suitable for in vivo imaging of the radioisotope. Such methods include PET and SPECT. By inspection of the imaging results, the clinician may determine the amount of myelination, as reflected by levels and anatomical localization of signal emitted by the radioisotope derivative of the agent and detected by the appropriate imaging methodology. In certain embodiments the agent is a radioisotope derivative of the compound of Formula I.

To determine whether myelination in the patient may be deficient, myelination levels may be compared to those exhibited by a subject or subjects believed or known not to be suffering from a myelin-associated neuropathy. In another embodiment, rates of demyelination or remyelination may be determined. Following treatment with a known or suggested therapeutic agent believed or anticipated to prevent or slow demyelination or to promote remyelination in patients suffering from myelin-associated neuropathies, myelination levels are evaluated by performing the imaging over time in the patients treated with the therapeutic agent. The imaging may be performed at different points of time and the level of myelination at one time point compared to that of another.

A positive result suggestive of a myelin-associated neuropathy may be one in which the decrease of myelin basic protein of the subject, compared to a baseline measurement of myelin basic protein, in a control sample is statistically significant. The control sample may be from a similar sample free of a myelin-associated neuropathy or from the same subject with measurements taken over time.

In yet another embodiment, a biopsied mammalian tissue sample, or a tissue sample cultured in vitro, may be contacted with an agent specific for binding to myelin basic protein. The agent may comprise the compound of Formula I, a ¹³C enriched compound of Formula I, or a ¹⁹F-labeled-derivative of Formula I. Contacting with the agent may be used to determine the location, presence, or amount of myelin basic protein in the tissue sample. The tissue sample may be sampled from a subject that has been experimentally manipulated so as to serve as a verified or purported model of myelin-associated neuropathy, or that has received at least one therapeutic agent verified as, or purported to be, a treatment for myelin-associated neuropathy. The therapeutic agent may be associated with the preclinical evaluation or basic neuroscience research aimed at studying the function and process of myelination, and the dysfunction and repair of myelin.

Fresh frozen cryostatic sections, or fixed or embedded sections or samples, of the biopsy or culture tissue sections, may be contacted with an agent specific for binding to myelin basic protein. The samples may be prepared using various sectioning techniques such as microtome, vibratome, or cryostat preparation. The agent may comprise the compound of Formula I, or a ¹³C enriched compound of Formula I, or an ¹⁹F-labeled-derivative of Formula I

After binding to myelin basic protein, the sample may be washed in a manner and medium suitable to remove any unbound and non-specifically bound label from the sample, without eliminating specific binding to myelin basic protein.

Any of a number of detection, visualization, or quantitation techniques, including but not limited to fluorescence microscopy, laser-confocal microscopy, cross-polarization microscopy, autoradiography, MRI, MRS, or other applicable methods, or any combination thereof, may be then be used to assess the presence or quantity of an agent having specific binding to myelin basic protein in the tissue sample and may represent the presence or amount of myelin basic protein. In certain embodiments, the agent may comprise the compound of Formula I, a ¹³C enriched compound of Formula I, or a ¹⁹F-labeled-derivative of Formula I. The labeling with, and detection, visualization, or quantitation of the an agent, may also be performed in conjunction with labeling with, and detection, visualization, or quantitation of at least one other compound that specifically binds a substance other than myelin basic protein.

### Examples

The following non-limiting Examples are shown and describe various embodiments of the present invention.

### Example 1

### Preparation of Nerve Tissue Sections

Various nerves including sciatic, femoral, brachial plexus, trigeminal, optic, and penile were harvested from male Sprague Dawley rats or male CD-1 mice. Tissue was fixed by perfusion and/or post-fixation with formalin. Following post-fixation, tissue was cryoprotected in a 20% sucrose solution made in phosphate buffered saline (PBS). Nerves were then flash-frozen using methanol and dry ice in OCT media. In some cases, PVDF membranes were used to help keep the nerves vertical in the OCT media. Thin sections (5-10 um) were sliced on a Leica microtome and stored in a −80° C. freezer prior to staining with antibodies or small molecule compounds.

### Example 2

### Histological Evaluation of Nerve Tissue Sections by Antibody

Some nerves were stained for hematoxylin and eosin in order to identify basic nerve morphology. Serial sections of the nerves were stained for a panel of myelin proteins; including myelin basic protein (MBP), myelin protein zero (MPZ), myelin associated glycoprotein (MAG), and peripheral myelin protein 22 (PMP22), and Schwann cell proteins 2′,3′-Cyclic Nucleotide 3′-Phosphodiesterase (CNPase) and S100. Antibody vendor, catalog number and dilutions are shown in Table I. The nerves were stained on an automated Ventana Discovery XT immunostainer (Roche). Non-paraffin tissues were pre-treated in Cell Conditioning Solution, CC1, (Ventana). The slides were then blocked in 10% serum (species determined by host of secondary antibody). The primary and secondary antibodies were applied via manual application and incubated with heat (37° C.) on the immunostainer for one hour with rinses in between. The slides were then removed from the immunostainer and rinsed in a Dawn dish detergent solution to remove the mineral oil from the slides. Slides were then coverslipped by Vectashield™ mounting media. All secondary antibodies were purchased from Jackson ImmunoResearch Laboratories and were either Cy3 or Cy5 conjugated and used at a dilution of 1:200. After cover slipping, the slides were imaged on a Zeiss Axioimager microscope at 20×, using the appropriate filter set for each secondary antibody.

TABLE I
Antibodies used in characterization of nerves

| Antibody | Vendor + Catalog # | Dilution |
|---|---|---|
| MBP | Abcam ab2404 | 1:50 |
| MPZ | Santa Cruz sc-18533 | 1:50 |
| MPZ | Abcam ab39375 | 1:100 |
| CNPase | Lab Vision/Thermo MS-349 | 1:50 |
| MAG | Millipore/Chemicon MAB1567 | 5-10 ug/mL |
| S100A1 | Lab Vision/Thermo MS-296 | 1:100 |
| PMP22 | Lab Vision/Thermo MS-1293 | 2-4 ug/mL |
| PMP22 | Abcam ab | 1:50 |

### Example 3

### Measurement of Optical Properties of the Small Molecule Fluorophores

The fluorophores agents were dissolved in dimethylsulfoxide (DMSO) to make a 10 mM stock solution. An aliquot was taken to prepare a 10 nm-1 uM fluorophore solution in methanol, water, or DMSO. Optical measurements from the three solvents were taken. Absorbance spectra were measured using a Perkin Elmer Lambda 20 UV/VIS spectrometer. Emission spectra were generated using a PTI steady state fluorimeter.

### Example 4

### Ex vivo Staining of Nerves by the Fluorophores

The fluorophores were dissolved in DMSO to make a 10 mM stock solution. Slides containing nerve tissue sections were rinsed three times with PBS. The tissue sections were incubated with a solution of 10 uM of each fluorophore diluted in either PBS or a mixture of 99 uL DMSO, 100 uL cremaphor, 600 uL rat serum, and 200 uL PBS for 20 minutes. The slides were then washed with PBS for 5 min three times, cover-slipped with Vectashield and imaged on a Zeiss Axioimager microscope at 200× magnification. A custom filter cube (excitation filter: 387 nm with 11 nm bandpass, 409 nm dichroic mirror; emission filter 409 nm long pass) was used to collect images for examination of morphology and for image analysis.

Co-staining of the nerves with the fluorophores and various myelin antibodies was also performed. These slides were stained on the Ventana Discovery XT using the same protocol described above with some modification. The fluorophore was added directly to the primary antibody solution for a final fluorophore concentration of 10 uM and a final antibody dilution from Table 1. The slides were imaged using the Zeiss Axioimager microscope at 20× and analyzed as follows: Raw tagged image format images were used in all cases. Within each image representing the fluorophore channel, several circular areas of interest were drawn representing nerve-containing tissues, adjacent tissues, and regions without tissues. All areas of interest were identical in size, and all regions of the image were represented. The identical, co-localized areas of interest were drawn in the secondary antibody channel. The average channel signal intensities from each areas of interest were plotted against each other. The secondary antibody channel was plotted on the X-axis and the agent channel was plotted on the Y-axis. Regression coefficients were then calculated.

### Example 5

### Isolation of Native Myelin Basic Protein from Rat Brain

Purified myelin basic protein from rat brain was used for further evaluation of fluorophore binding. Crude myelin was isolated using a modified procedure from Current Protocols in Cell Biology (2006) 3.25.1-3.25.19. Isolation of native myelin basic protein from crude myelin was performed following the protocol from NeuroReport 5 (994) 689-692. Briefly, three rat brains from male Sprague Dawley rats were dissected, placed in 72 ml cold 0.30 M sucrose solution, diced and homogenized. The homogenate was layered over an equal volume of a 0.83 M sucrose solution, subjected to ultracentrifugation at 75,000 g at 4° C. for 30 min, and crude myelin collected at the interface of the two sucrose solutions.

The collected myelin fraction was subjected to osmotic shock by homogenization in Tris-Cl buffer (containing 20 mM Tris-Cl, pH 7.45, 2 mM sodium EDTA, 1 mM dithiothreitol, and protease inhibitor cocktail). Additional Tris-Cl was added to a final volume of 228 ml. The suspension was centrifuged at 75,000×g, 4° C. for 15 min. The pellet was subjected to two more times of homogenization and ultracentifugation at 12,000 g, 4 C for 15 min each time. The pellet was resuspended in 72 ml of 0.3 M sucrose solution. An equal volume of 0.83 M sucrose solution was layered over the resuspended pellet and the entire sample subjected to ultracentrifugation at 75,000 g at 4 C for 30 min. Purified myelin was collected from the interface, and resuspended in 228 ml Tris-Cl buffer. Washout of excess sucrose was performed by additional homogenization in Tris-Cl buffer and centrifugation as described above.

The myelin pellet was resuspended in 5 volumes of Buffer 1 (containing cold 500 mM NaCl/20 mM Tris-HCl/2 mM B-mercaptoethanol, pH 8.5) for 30 min, and then centrifuged on a JA20 at 15,000 rpm for 20 min. This was repeated twice. The pellet was solubilized into 2% CHAPS solution, incubated on ice for 30 min, then centrifuged at 40,000 rpm for 45 min, on Beckman 42.1 rotor. The CHAPS extract was loaded onto a hydroxyapatite column (1.6×5 cm) that was pre-equilibrated with 1% CHAPS solution. Lipid-bound MBP was eluted in the non-adsorbed pass-thru fraction. The pass-thru fraction was concentrated using an Amicon filter YM3. The concentrate was loaded onto a spectra gel AcA 44 gel filtration column that was pre-equilibrated with Buffer 2 (containing 1% CHAPS, 20 mM Tris-Cl, pH 8.5, 1 mM beta-mercaptoethanol, 1 mM dithiothreitol, 0.5 mM EDTA, 0.5 mM EGTA, 1 mM 1,10 phenanthroline, 1 mM zinc acetate). The lipid-bound MBP was concentrated, salted out using 50% ammonium sulfate, lyophilized, and stored under nitrogen at 4° C. The samples were run in a standard denaturing polyacrylamide gel electrophoresis and Western blot. Reagents and standards for gel electrophoresis were from Invitrogen. Commercially available mouse MBP (Sigma) was used as a control.

### Example 6

### Fluorophore Binding to Isolated Native Myelin Basic Protein

Spectramax fluorescent assay: 0.5 nmol of the fluorophore was pipetted into a low-fluorescence 96-well plate. Using a Spectramax M5 multi-modality plate reader (Molecular Devices), the absorbance was scanned as well as the emission properties when excited at the peak absorbance wavelength. 0.5 nmol each of native MBP, and denatured MBP was added to the fluorophore, and the absorbance and emission properties of the fluorophore were re-measured.

### Example 7

### In vivo Imaging

CD-1 mice (25-40 g), housed in an AAALAC-compliant facility, were weighed and anesthetized by induction and maintenance on 2.5% Isoflorane. Animals were placed on their backs on a warming pad. With one hand the skin was held taut while 50 uMoles/kg of the agent to be evaluated (100% DMSO and centrifuged at 10,000 g for 20 min) was injected intraperitoneally or intravenously with a 300 ul syringe equipped with a 30 gauge needle. The animals were allowed to recover from anesthesia and assume normal activity for four hours. At that time they were then anesthetized by induction and maintenance on 2.5% Isoflorane. They were injected as above with 100 ul of Fatal Plus (pentobarbital). The thoracic cavity and abdomen were accessed. The inferior vena cava was severed and 12 ml of phosphate buffered saline was infused via cardiac puncture at approximately 1 ml per minute followed by 12 ml of phosphate buffered formalin. Key nerves were exposed and imaged using a Zeiss Lumar V.12 surgical microscope equipped with filter sets appropriate for the fluorophore.

### General Synthetic Schemes and Procedures:

Formula I (R1=CH3, R2=NH2 and R3=CH3) was prepared according to the transformations outlined in Scheme 1. Preparation of aldehyde 3 and phosphonate 5 have been described in U.S. patent application Ser. No. 12/478,300.

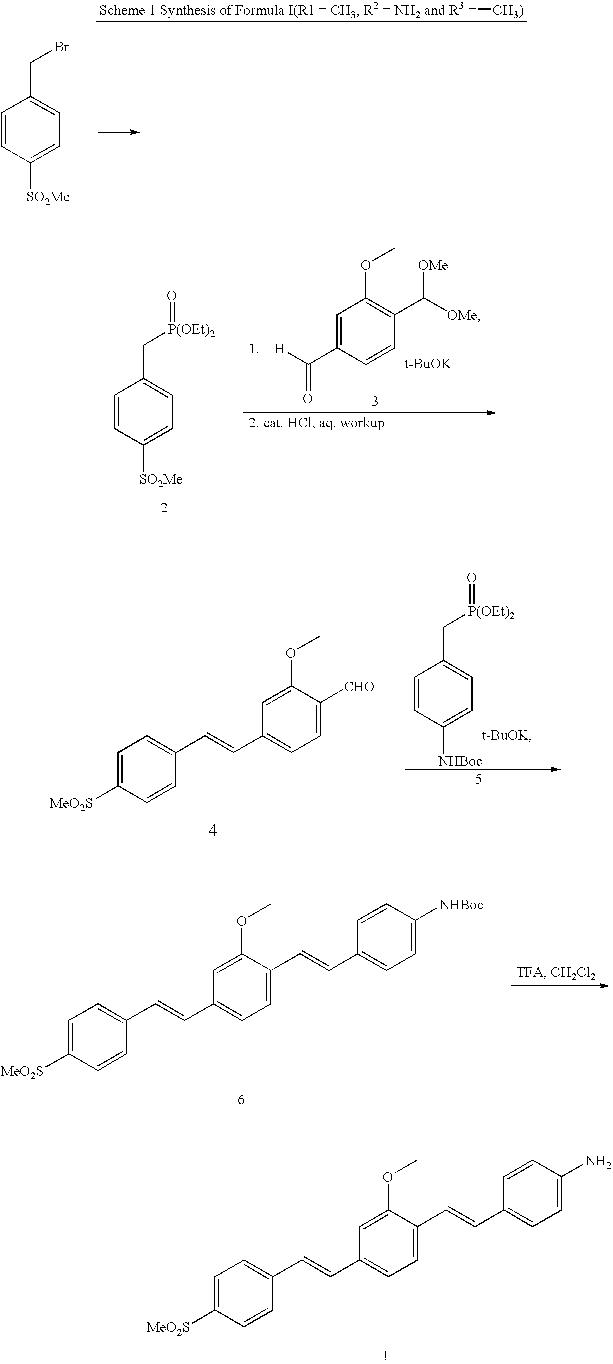

### Diethyl 4-methylsulfonylbenzyl phosphonate 2b

A solution of 4-methylsulfonyl benzyl bromide 2a (1 g, 4 mmol) in triethylphosphite (2.8 ml, 16 mmol) was warmed up to 100 C for 2 hrs. GC-MS indicated complete conversion. The mixture was devolatilized under vacuum, to give the desired product as a light yellow oil (1.22 g, 99%). GC-MS(EI+): 306(M+), 278, 263, 250, 227, 199, 183, 170, 124, 109, 107(100%), 104, 97, 90.

### (E)-2-methoxy-4-(4-(methylsulfonyl)styryl)benzaldehyde 4

To a dry vial containing phosphonate 2 (579 mg, 1.89 mmol) under N2 was added dry THF (4 ml) followed by a solution of t-BuOK (250 mg, 2.268 mmol) in 3 ml dry THF. After stirring for 5 min. at room temperature, a solution of the aldehyde 3 in 3 ml dry THF was added dropwise and the mixture was stirred at 60 C bath temperature for 2 hrs. The reaction volume was reduced under a n2 stream, ethyl acetate and brine was added, and the pH of the aqueous phase was brought to 3 with dilute (0.1N) HCl. The mixture was shaken, the phases were separated and the aqueous phase was extracted with ethyl acetate (2×). The combined organic phases were dried over Na₂SO₄. The drying agent was filtered off, silicagel 60 was added and the compound was adsorbed on silicagel and purified by MPLC using dichloromethane-ethyl acetate gradient 5-30% v/v. Yellow solid, 504 mg (74%). LC-MS(ESI+): 317 (M+H+), 358 (M+CH3CN+H+). NMR (CD2Cl2): 10.46 (s, 1H), 7.97 (2H, dd, J=8.2, 0.8 Hz), 7.85 (1H, J=8.6 Hz), 7.78 (2H, dd, J=8.2, 0.8 Hz), 7.35 (2H, d J=0.8 Hz), 7.28 (1H, d, J=12.6 Hz), 7.21 (1H, d J=0.8 Hz), 4.04 (3H,s), 3.09 (3H,s).

### tert-Butyl 4-(2-methoxy-4-(4-(methylsulfonyl)styryl)styryl)phenylcarbamate 6

Because of the poor solubility of the aldehyde 4 in THF, a modified olefination procedure was employed, as follows: to a dry vial containing phosphonate 5 (105 mg, 0.3075 mmol) in dry THF (1 ml) under N₂ was added a solution of t-BuOK (40.3 mg, 0.36 mmol) in 0.25 ml THF followed by a 0.25 ml THF rinse of the t-BuOK vial. The blue-colored mixture was stirred under N2 at r.t. for 5 min, and then added to a solution-suspension of aldehyde 4 in dry THF (1 ml) under N₂, vial canula. Upon completion of the addition, the brick-red solution was stirred at 62 C bath temperature for 1 hr. LC-MS at this point indicated a very clean and complete conversion to the desired product. The product has poor solubility in most common solvents, except THF. The reaction mixture was rotovapped dry and any excess base was neutralized with a small piece of dry ice and the solid was left under a blanket of CO₂ overnight. It was then dissolved in THF, adsorbed on silicagel and purified by MPLC using hexanes-THF gradient 40-80%. The compound elutes at 60% v/v THF as a light orange solid (124 mg (83%). MS (ESI+): 505 (M+), 528 (M+Na+). H-NMR (acetone-D6): 8.47 (1H, s), 7.93 (2H, d, J=8.5 Hz), 7.85 (2H, d, J=8.5 Hz), 7.68 (1H, d, J=8 Hz), 7.57 (2H, d, J=8.5 Hz), 7.51 (2H, d, J=8.1 Hz), 7.47 (1H,s), 7.42 (2H, dd, J=12, 3.2 Hz), 7.34 (1H, s), 7.28-7.23 (2H, m), 3.98 (3H,s), 3.12 (3H,s), 1.49(9H,s). C-NMR (acetone-D6): 158.21, 153.81, 143.82, 140.39, 138.16, 133.27, 130.02, 128.83, 128.01, 127.59, 127.32, 122.03, 121.01, 119.35, 80.26, 56.20, 44.56, 28.69.

### 4-(2-Methoxy-4-(4-(methylsulfonyl)styryl)styryl)aniline (Formula I: R1=CH₃, R²═NH₂ and R³═—CH₃):

To a solution of Boc-3111 (6, 16.4 mg, 32.4 mol) in dichloromethane (0.8 ml) containing 40 ppm amylene was added TFA (0.2 ml) and the mixture was stirred at room temperature for 30 minutes. LC-MS analysis indicated a very clean and complete deprotection. The solvent was stripped with a stream of N₂, the compound was dissolved in 0.2 ml THF, and adsorbed on a silica SPE cartridge. Following initial elution with hexanes, addition of 50 l of triethylamine and elution with THF produced the desired dye, 10.5 mg (81%) as a dark red solid MS(ESI+): 406 (M+H+), 447 (M+CH3CN+H+).

Preparation of several other intermediates, two of which, phosphonate 8 and aldehyde 9, isomeric with aldehyde 3, have been prepared according to the synthetic transformation outlined in Scheme 2.

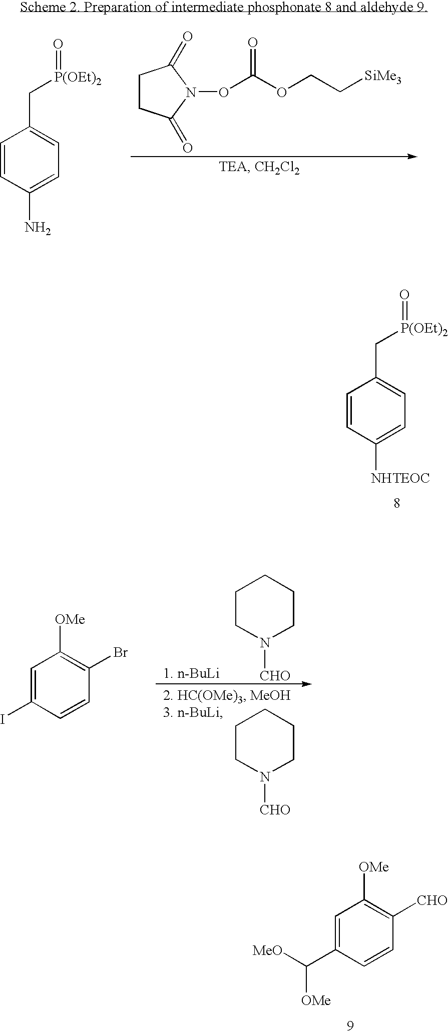

### 2-(Trimethylsilyl)ethyl 4-((diethoxyphosphoryl)methyl)phenylcarbamate 8

To a solution of diethyl 4-aminobenzylphosphonate (922 mg, 3.8 mmol) in dichloromethane (12.6 ml) was added triethylamine (2.66 ml, 19 mmol). The mixture was stirred for 5 minutes, then succinimidyl-TEOC (985 mg, 3.876 mmol) was added in one portion and the mixture was stirred at room temperature for 40 hrs. The solution was washed with brine (3×), dried over Na2SO4, adsorbed on silicagel and purified by MPLC using hexanes-ethyl acetate 50-100% gradient. Colorless oil solidifying at low temperature to a wax. Yield: 822 mg (56%). Note: the trailing fraction yielded additional 522 mg product of less than 99% purity. MS(ESI+): 388 (M+H+), 410 (M+Na+). NMR(CD2Cl2): 7.39(2H, d, J=8.3 Hz), 7.23 (2H, dd, J=17.4, 2.2 Hz), 4.24-4.28 (2H, m), 4.02-4.06 (2H,m), 3.12 (2H, d, J=23.2 Hz), 1.28(6H, J=7.2 Hz), 1.06 (2H,m), 0.1(9H,s). C-NMR(CD2Cl2): 153.76, 137.51 (d, J=3.7 Hz), 130.16 (d, J=6.6 Hz), 126.12 (d, J=8.8 Hz), 118.61, 63.18, 62.03 (d, J=6.6 Hz), 33.47, 32.10, 17.68, 16.19(d, J=5.8 Hz), −1.86.

### 4-bromo-3-methoxybenzaldehyde

A solution of 2-bromo-5-iodoanisole (5 g, 16 mmol) and a crystal of 1,10-phenanthroline (indicator) in dry Et2O (45 ml) was cooled to −78 C in a dry ice-acetone bath. A solution of n-BuLi (2.5M in hexanes) was added dropwise until the end-point was reached (7.8 ml). The mixture was stirred at this temperature for 15 minutes, during which time period a thick slurry formed. To the suspension was added dry N-formylpiperidine (3.46 ml, 31.2 mmol) via syringe and the mixture was slowly allowed to reach room temperature over 30 min. GC-MS at this point indicated no aryl iodide. The reaction mixture was washed with 1 N HCl (2×), brine (once), the aqueous phases were extracted with ether, the combined organic phases were dried over Na2SO4, and the solvent was removed on rotovap, yielding a light yellow oil which was taken directly to the next step. Note: an aliquot yielded a white crystalline product upon washing with a small amount of cold methanol. MS(EI+): 216(M+,100%), 214(M+, 100%), 215, 213, 201, 199, 187, 185, 172, 170, 157, 155, 145, 143, 119, 105, 92, 77, 63.

### 4-(Dimethoxymethyl)-2-methoxybenzaldehyde 9

The crude aldehyde above (3.4 g, 15.8 mmol) was dissolved in methanol (62 ml) and trimethylorthoformate (17 ml, 158 mmol). Para-toluenesulfonic acid monohydrate was added (300 mg, 0.158 mmol) and the mixture was refluxed for 3 hrs. Upon cooling to room temperature, a spatula of solid NaHCO3 was then added, the mixture was stirred for 10 min. adsorbed on silicagel and purified by MPLC eluting with hexanes-ethyl acetate (20-60% EtOAc). Yield: 3.81 g (92%) light yellow oil, which was taken to the next step. MS(EI+): 262(M+), 260(M+), 231(100%), 229(100%), 216, 215, 214, 213, 122, 75.

To a solution of the aryl bromide-acetal above (3.812 g, 14.6 mmol) and a crystal of 1,10-phenanthroline (indicator) in dry ether (41 ml) at −78 C (acetone-dry ice bath) was slowly added a solution of n-BuLi in hexanes (2.5M) until equivalence (6.3 ml). After 5 minutes, the dry ice-acetone bath was replaced with an acetonitrile-dry ice bath and the mixture was stirred for 45 minutes at −40 C internal temperature. At this point N-formylpiperidine (3.16 ml, 28.47 mmol) was added via syringe and the mixture was allowed to warm up to room temperature over 1 hr. Water was then added carefully, the organic layer was washed with water (3×), brine (once), the aqueous layers were extracted with ether and the combined organic phases dried (Na2SO4) and the crude product was purified by MPLC eluting with hexanes/ethylacetate (5-40% then 60% v/v EtOAc). Yield: 2.604 g (85%) colorless oil. MS(EI+): 210(M+), 179(100%), 163, 151, 135, 119, 108, 91, 75.

Formula I (R1=CH₃, R²═NH₂ and R³═—F₃) was prepared according to the transformations outlined in Scheme 3. Although a Boc-protected amino aldehyde may be used, a TEOC-protected aminoaldehyde 10 instead.

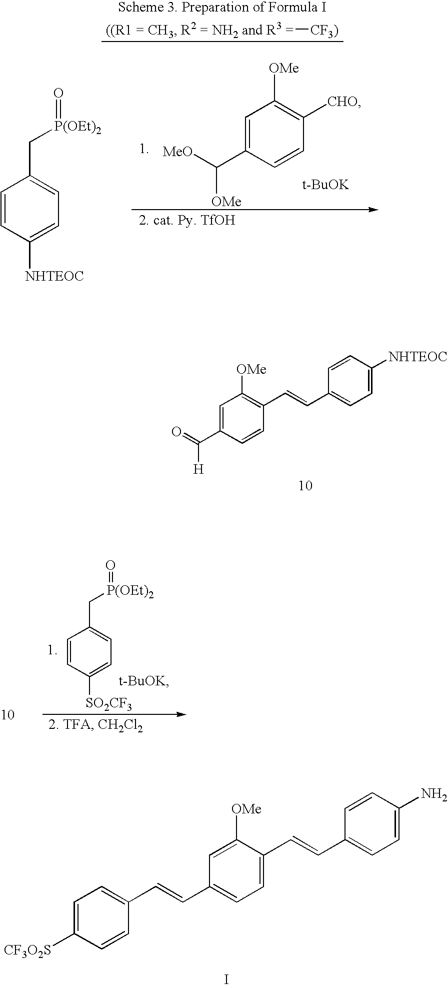

The required diethyl-4-trifluoromethylsulfonylbenzylphosphonate was prepared according to the sequence below:

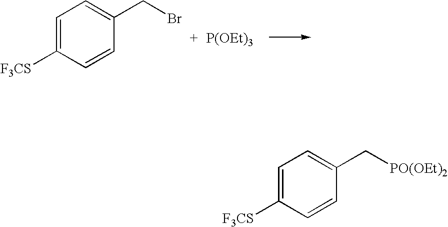

To 5.11 g of benzyl bromide was added 12 ml of triethylphosphite. The resulting solution was heated at 80° C. for 4 hours. The reaction mixture was concentrated under a flow of nitrogen and then purified on a large silica gel column (˜250 ml of silica) eluting with 80/20 hexanes/CH₂Cl₂ with increasing proportions of CH₂Cl₂ and finally adding in MTBE to elute the product. Yield was quantitative. 1H NMR (CDCl₃): 7.61 ppm (2H, d, J=8.0 Hz), 7.37 ppm (2H, dd, J=8.3, 2.4 Hz), 4.04 ppm (4H, dq, J=1.5, 7.1 Hz), 3.18 ppm (2H, d, J=22 Hz), 1.30 ppm (6H, t, J=7.1 Hz). 13C NMR (CDCl3): 136.5 ppm (d, J=2.9 Hz), 135.2 ppm (d, J=9.5 Hz), 130.9 ppm (d, J=6.6 Hz), 129.5 ppm (dq, J=2.9, 307 Hz), 122.8 ppm (m), 62.3 ppm (d, J=6.6 Hz), 33.7 ppm (d, J=138 Hz), 16.3 ppm (d, J=6.6 Hz).

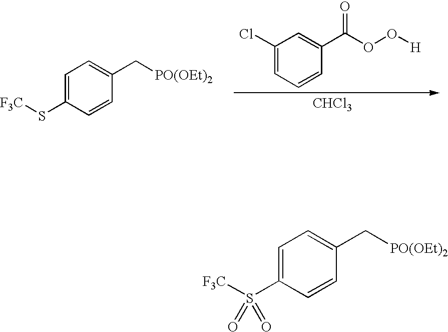

To 0.50 g of sulfide in 5 ml of CHCl3 was added 0.266 g of MCPBA; the reaction mixture was stirred at room temperature for 60 h. An aliquot analyzed via HPLC indicated 2 major peaks. An additional 0.060 g of MCPBA was added and the reaction stirred for 24 h. The reaction mixture was concentrated under nitrogen, treated with 15 ml MTBE and extracted with ˜6 ml and a further ˜4 ml of 0.8 M NaHCO+. The organic layer was dried with MgSO₄, filtered and concentrated. It was purified on an ISCO prep system using a silica gel column and a gradient starting at 100% CH2Cl2 and ending with 100% MTBE. Yield was quantitative. 1H NMR (CDCl3): 7.98 ppm (2H, d, J=8.2 Hz), 7.61 ppm (2H, dd, J=2.3, 8.5 Hz), 4.06 ppm (4H, dq, J=8.1, 7.1 Hz), 3.28 ppm (2H, d, J=22.5 Hz), 1.25 ppm (6H, t, J=7.1 Hz). 13C NMR (CDCl3): 142.2 ppm (d, J=9.3 Hz), 131.3 ppm (d, J=6.1 Hz), 130.9 ppm (d, J=2.2 Hz), 129.6 ppm (m), 119.7 ppm (q, J=325.8 Hz), 62.5 ppm (d, J=7.0 Hz), 34.3 ppm (d, 137.1 Hz), 16.3 ppm (d, J=5.9 Hz).

### (E)-2-(trimethylsilyl)ethyl 4-(4-formyl-2-methoxystyryl)phenylcarbamate 10

To a dry vial containing phosphonate 8 (537.5 mg, 1.346 mmol) was added dry THF (3 ml) followed by a solution of t-BuOK (180 mg, 1.607 mmol) in THF (2 ml) and the mixture was stirred at r.t. for 5 minutes. A solution of aldehyde 9 (278 mg, 1.32 mmol) in THF (2 ml) was then added dropwise and the mixture was stirred under N2 at 64 C bath temperature for 2 hrs. The mixture was chilled with ice, and the pH was adjusted to 5.5 with NaHSO4 then to 7 with NaHCO3, saturated brine was added and the mixture was extracted with ethyl acetate (4×). The solvent was removed on rotovap and the resulting oil was dissolved in THF 925 ml) and water (5 ml). A catalytic amount of pyridinium triflate (3 mg) was added and the mixture was stirred at 60 C for 75 minutes. Upon addition of solid NaHCO3 (50 mg) and stirring for 5 minutes, the solution was evaporated to dryness on rotovap (final pressure 10 torr), the yellow solid adsorbed on silicagel and purified by MPLC (hexanes-THF). MS(ESI+): 397(100%, M+), 398(28%), 299(6%). H-NMR(CD2Cl2): 9.98(1H,s), 7.79(1H,d, J=7.8 Hz), 7.56(2H, d, J=8.6 Hz), 7.50 (2H, dd, J=9.2, 0.9 Hz), 7.44-7.47(3H, m), 7.28(1H, d, J=16.6 Hz), 6.81(1H,br s), 4.27-4.32 (2H, m), 4.00 (3H, s), 1.08-1.12(2H, m), 0.11(9H,s).

### 2-(Trimethylsilyl)ethyl 4-(2-methoxy-4-(4-(trifluoromethylperoxythio)styryl)styryl)phenylcarbamate

To a solution of diethyl 4-trifluoromethylsulfonylbenzyl phosphonate (91.3 mg, 0.253 mmol) in dry THF (0.25 ml) was added a solution of t-BuOK (31 mg, 0.277 mmol) in THF (0.25 ml, followed by a 0.25 ml rinse) and the solution was stirred at r.t. for 5 minutes. A solution of aldehyde 10 (98.5 mg, 0.248 mmol) in THF (1 ml, followed by 2×0.25 ml rinse) was then added and the solution was stirred under N2 at 60 C for 90 minutes. The mixture was diluted with THF and carefully neutralized with powdered dry ice. The crude mixture was then adsorbed on silicagel and purified by MPLC on a stack of 12 g Gold Label columns using hexanes-THF gradient 30-40% THF. Yield: 106 mg (69.5%). MS (ESI+): 604(M+H+).

### 4-(2-Methoxy-4-(4-(trifluoromethylperoxythio)styryl)styryl)aniline Formula I ((R1=CH₃, R²═NH₂ and R³═—CH₃))

To a cold (° C.) solution of TEOC-11 above (10.7 mg, 17.75 mol) in dichloromethane (0.8 ml) containing 40 ppm amylene was added TFA (0.2 ml) dropwise and the mixture was allowed to slowly warm up to r.t. over cca 30 minutes and stirred for a total of 90 minutes. LC-MS indicated complete and very clean conversion to the desired product. The volatiles were stripped off with a stream of nitrogen and the dark residue was redissolved in dichloromethane, washed with a saturated aq. NaHCO3 solution, dried and the solvent stripped again with a stream of N2 to give the clean (99% integration) product as a dark orange powder (7.5 mg, 92% yield). MS (ESI+): 460(M+H+). H-NMR(CD2Cl2): 8.03 (2H, d, J=8.5 Hz), 7.83(2H, d, J=8.5 Hz), 7.64(1H, d, J=8.1 Hz), 7.38-7.42 (3H, m), 7.32 (1H, d, J=16.4 Hz), 7.21-7.25(1H, dd J=8.1 Hz, 1.5 Hz, flanked by 1H,d, J=16.4 Hz), 7.14(1H,m flanked by 1H, d, J=16.4 Hz), 6.71(2H, d, J=8.3 Hz), 5.36 (1H, m, J=1 Hz), 3.99 (3H,s).

A radioisotope derivative of the compound of Formula I may be prepared and imaging accomplished through radioimaging. Alternatively, a ¹³C enriched compound of Formula I or a ¹⁹F-labeled derivative of Formula I may be prepared. In certain embodiments, a compound of formula I having R1=CH2CH2OTs (where Ts is tosylate) may be used as precursor for radiolabeling with ¹⁸F (PET); other choices for the tosylate leaving group may be selected as generally known in the radiolabeling practice. Additionally, a compound of Formula I where R1 or R3=CF3 or C1-C4 perfluoroalkyl may be used for ¹⁹F-based MRI and a compound of Formula I where R1 or R3=¹³C-methyl or ¹³C-enriched C1-C4 alkyl may be used for ¹³C-based MRI.

Alternatively, a ¹³C labeled derivative of the compound of Formula I may be prepared by alkylating the amino functionality of the compound of Formula I with ¹³C enriched methyl iodide or a similar C1-C4 alkylating agent. A ¹⁹F derivative of the compound of Formula I may be prepared by alkylating the amino functionality of the compound of Formula I with a C1-C4 fluoro- or perfluoroalkyl halide, mesylate, or tosylate, by reacting with a fluoroacyl halide such as pentafluorophenyl benzoyl chloride to yield the corresponding amide or by reductive amination where the carbonyl component bears a C or ¹⁹F moiety. In other embodiments, the amine moiety of Formula I may be alkylated to produce a 2-hydroxyethyl derivative which can be used via its tosylate or mesylate as a precursor for the radiolabeling with ¹⁸F (PET).

### Results and Observations

TABLE II
Fluorescence excitation and emission peaks of Select Compounds

| | | | | Nerve binding, ex | Excitation | Emission | Excitation | Emission | Excitation | Emission |
|---|---|---|---|---|---|---|---|---|---|---|
| Formula | R1 | R2 | R3 | vivo | (MeOH) | (MeOH) | (CH₂Cl₂) | (CH₂Cl₂ | (DMSO) | (DMSO) |
| I | CH₃ | NH₂ | CH₃ | +++ | 377 | 450 | — | — | 412 | 630 |
| I | CH₃ | NH₂ | —CF₃ | +++ | 407 | 500 | 413 | 610 | 433 | 500 |

Fluorescence excitation and emission peaks of various compounds, and relative binding are shown in Table II. A +++ indicates binding to nerves using the ex vivo histochemical assay.

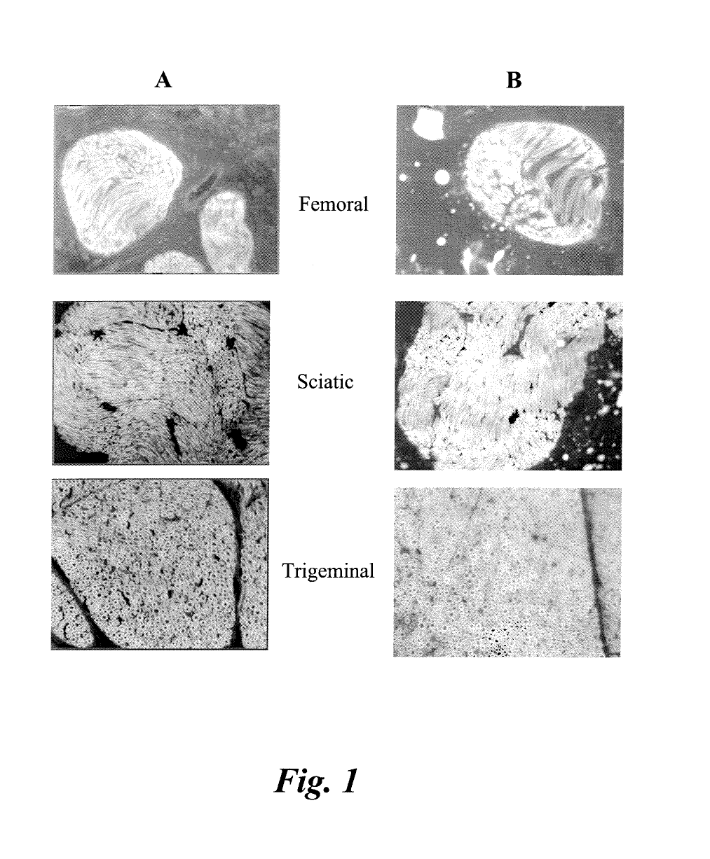

Examination of the hematoxylin and eosin staining of nerve tissue sections revealed characteristic nerve morphology can be identified. Each nerve or nerve bundle appeared as a large circle or group of large circles within which smaller donut-shaped myelinated axons can be identified. The nerve sections were stained with the fluorophores. FIG. 1 shows staining of the trigeminal, sciatic, and femoral nerves with fluorophores. As shown, the myelinated donut-shaped structures are visible. The control slides containing the nerves with no agent (not shown) was negative under the same imaging conditions.

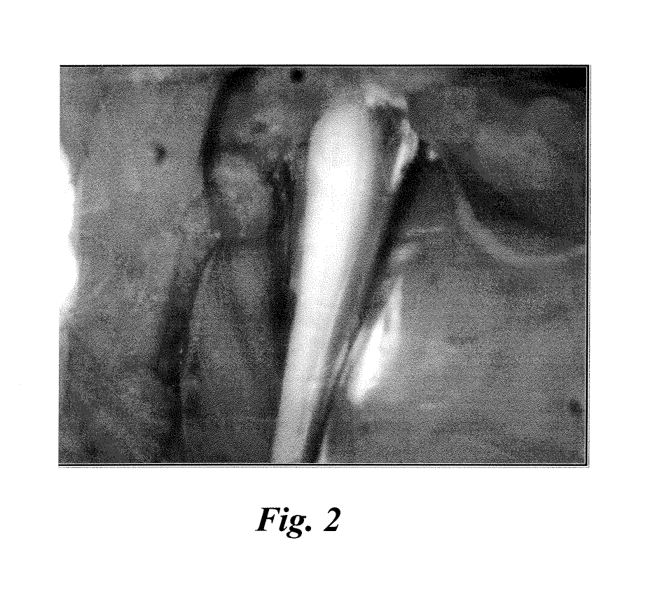

When the agents were injected systemically to the pre-clinical animal model, in vivo imaging revealed that some of the agents localized to nerves in a number of tissues including the brachial plexus, facial nerve, trigeminal nerve, phrenic nerve, vagus nerve and optic nerve when administered systemically to a pre-clinical animal model. The adjacent muscle tissues had very low background binding. The nerves of the negative control animals, with no fluorophore administered, had no fluorescent signal. FIG. 2 shows fluorescent in vivo imaging of brachial plexus nerves in the mouse surgical model by the Formula I (R¹═CH₃, R²═NH₂ and R³═CH₃). In vivo performance of the agents is a combination of several factors, including but not limited to agent myelin-binding property, blood nerve barrier penetration, metabolism, plasma binding, half-life, solubility, and clearance rate. Agents that did not stain nerve tissue sections in the ex vivo assay were typically not tested in vivo.

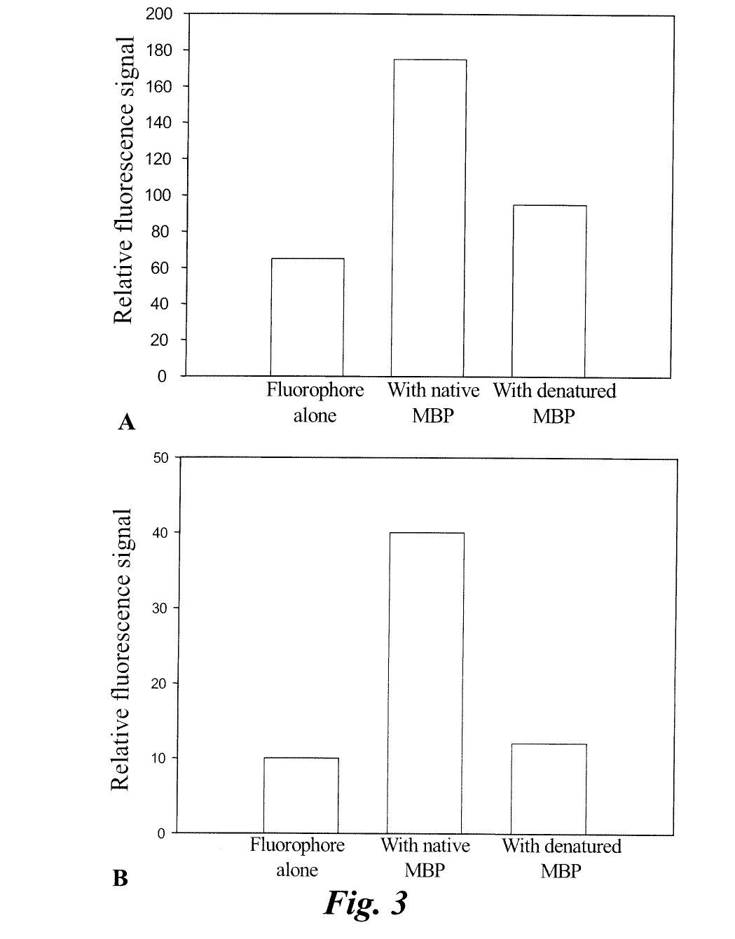

Native myelin basic protein was purified from rat brain and used in biochemical assays. Native MBP altered the fluorescence properties of Formula I wherein R¹═CH₃, R²═NH₂ and R³═CH₃ or CF₃ suggesting a close interaction between the fluorophore and MBP. FIG. 3 shows that the fluorescence emission intensity of Formula I (R¹═CH₃, R²═NH₂ and R³═—CH₃ or —CF₃) were enhanced upon binding to native MBP. Binding to denatured MBP did not result to significant enhancement in fluorescent intensity. The conjugation through the π double bond orbitals of the benzene rings and olefinic substituents may provide a path for electrons to flow from the electron-donating group R² to the electron-donating group R³across Formula I. This electron flow may contribute to a more pronounced enhancement of the fluorescent signal.

The invention may be embodied in other specific forms without departing from the spirit or essential characteristics thereof. The foregoing embodiments are therefore to be considered in all respects as illustrative rather than limiting on the invention described herein. The scope of the invention is thus indicated by the appended claims rather than by the foregoing description, and all changes that come within the meaning and range of equivalency of the claims are therefore intended to be embraced therein.

1. A method of detecting myelin-associated neuropathy comprising:
identifying a subject at risk of or diagnosed with a myelin-associated neuropathy;
administering to a subject an agent, wherein the agent comprises
a compound of Formula I, a ¹³C enriched compound of Formula I, an ¹⁹F-labeled derivative of Formula I, or a radioisotope derivative of Formula I; 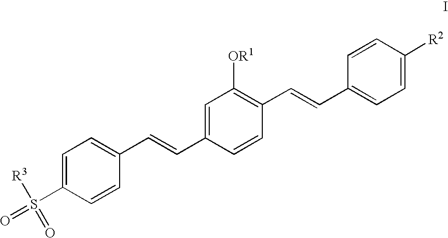
wherein R¹ is an alkyl group;
R² is an electron donating group; and;
R³ is an is an alkyl, substituted alkyl, amine or substituted amine;
determining myelination in the subject by detecting the agent present in the subject; and
comparing the myelination in the subject with a control sample wherein a lower level of agent in the subject is indicative of a myelin-associated neuropathy.

2. The method of claim 1 wherein R¹ is a lower alkyl group of from 1 to 6 carbon atoms and R2 is a primary amine, secondary amine, tertiary amine, or alkoxy.

3. The method of claim 1 wherein R3 is CH₃ or CF₃.

4. The method of claim 1 wherein the administration comprises intravenous injection, intraperitoneal injection, subcutaneous injection, intramuscular injection, intrathecal injection, intracerebral injection, intracerebroventricular injection, intraspinal injection, or combinations thereof.

5. The method of claim 1 wherein the detecting is effected by gamma imaging, MRI, MRS, PET, CEST, PARACEST, or a combination thereof.

6. The method of claim 1 wherein the detecting is effected by:
applying a light source, tuned to the spectral excitation characteristics of the compound of Formula I; and
observing the subject through an optical filter tuned to the spectral emission characteristics of the compound of Formula I.

7. The method of claim 1 further comprising the step of quantifying the amount of the agent in the subject.

8. The method of claim 8wherein the quantifying step comprises measuring radioactivity of the agent and wherein the agent comprises the radioactive derivative of Formula I bound to the tissue sample.

9. The method of claim 1 wherein the myelin-associated disease comprises multiple sclerosis, Guillain-Barré syndrome, leukodystrophies metachromatic leukodystrophy, Refsum' s disease, adrenoleukodystrophy, Krabbe's disease, phenylketonuria, Canavan disease, Pelizaeus-Merzbacher disease, Alexander's disease, diabetic neuropathy, chemotherapy-induced neuropathy, or a combination thereof.

10. A method of imaging myelin basic protein in a surgical field comprising the steps of:
contacting the surgical site with an agent, wherein the agent comprises a compound of Formula I, a ¹³C enriched compound of Formula I, an ¹⁹F-labeled derivative of Formula I, or a radioisotope derivative of Formula I; 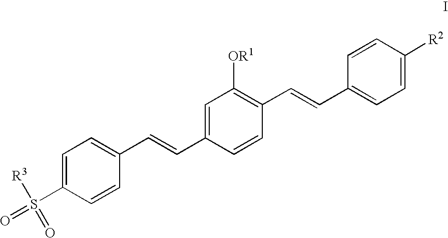
wherein R¹ is an alkyl group;
R² is an electron donating group; and;
R³ is an is an alkyl, substituted alkyl, amine or substituted amine; and
detecting the agent.

11. The method of claim 10 wherein R¹ is a lower alkyl group of from 1 to 6 carbon atoms and R2 is a primary amine, secondary amine, tertiary amine, or alkoxy.

12. The method of claim 10 wherein R³ is CH₃ or CF₃.

13. The method of claim 10 wherein the detecting step comprises:
applying a light source, tuned to the spectral excitation characteristics of the compound of Formula I, to the surgical field; and
observing the surgical field through an optical filter tuned to the spectral emission characteristics of the compound of Formula I.

14. A method of quantifying the amount of myelin present in a tissue sample comprising:
contacting the tissue sample with an agent wherein the agent wherein the agent comprises a compound of Formula I, a ¹³C enriched compound of Formula I, an ¹⁹F-labeled derivative of Formula I, or a radioisotope derivative of Formula I; 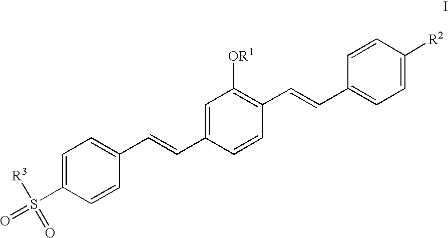
wherein R¹ is an alkyl group;
R² is an electron donating group; and;
R³ is an is an alkyl, substituted alkyl, amine or substituted amine; and
quantifying the amount of the agent present in the tissue sample by comparing to a baseline measurement of myelin basic protein in a control sample.

15. The method of claim 14 wherein R¹ is a lower alkyl group of from 1 to 6 carbon atoms and R² is a primary amine, secondary amine, tertiary amine, or alkoxy group.

16. The method of claim 14 wherein R³ is CH₃ or CF₃.

17. The method of claim 14 wherein the detecting is effected by fluorescence microscopy, laser-confocal microscopy, cross-polarization microscopy, autoradiography, magnetic resonance imaging, magnetic resonance spectroscopy, or combination thereof.

18. A kit for detecting myelin-associated neuropathy in a subject comprising:
an agent wherein the agent comprises a compound of Formula I, a ¹³C enriched compound of Formula I, an ¹⁹F-labeled derivative of Formula I, or a radioisotope derivative of Formula I; 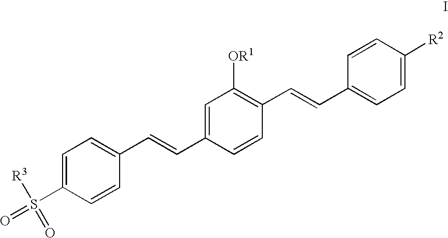
wherein R¹ is an alkyl group;
R² is an electron donating group; and;
R³ is an is an alkyl, substituted alkyl, amine or substituted amine; and
a pharmaceutically carrier.

19. The method of claim 18 wherein R¹ is a lower alkyl group of from 1 to 6 carbon atoms and R² is a primary amine, secondary amine, tertiary amine, or alkoxy.

20. The method of claim 18 wherein R³ is CH₃ or CF₃.
